(19) United States
(12) Patent Application Publication
(10) Pub. No.: US 2003/0187017 A1
(43) Pub. Date: Oct. 2, 2003

(54) Method for treating functional dyspepsia

(76) Inventor: Allen Wayne Mangel, Chapel Hill, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/276,238

(22) PCT Filed: May 15, 2001
(86) PCT No.: PCT/US01/15633

(51) Int. Cl. A61K 31/4745

(52) U.S. Cl. 514/292

(57) ABSTRACT

This invention relates to the use of alosetron in the treatment or functional dyspepsia.

The invention relates to a new medical use for alosetron, a compound which act as an antagonist of 5-hydroxytryptaminie (5-HT) at 5-HT3 receptors.

U.S. Pat. No. 5,360,800 and European Patent No. 0 306 323, incorporated herein by reference, disclose inter alia 2,3,4,5-tetrahydro-5-methyl-2-[(5-methyl-1H-imidazol-4-yl)methyl]-1H-pyrido[4,3-b]indol-1-one, now known as alosetron, which may be represented by the formula (I):

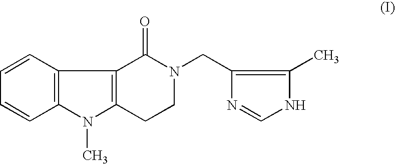

and pharmaceutically acceptable salts, solvates and pharmaceutically acceptable equivalents thereof; in particular its hydrochloride salt.

Alosetron is known to be useful for the treatment of a variety of conditions, including irritable bowel syndrome. PCT Publication No. WO 99/17755, published Apr. 15, 1999, incorporated herein by reference, in particular discloses the use of alosetron and other 5-HT₃ receptor antagonists for the treatment of irritable bowel syndrome in females. It has now been discovered that alosetron is useful for the treatment of functional (or “nonulcer”) dyspepsia.

Functional dyspepsia is a distinct type of dyspepsia. The term “dyspepsia” is defined as the general condition of indigestion and as such encompasses a variety of distinct dyspeptic conditions. There are several recognized types of dyspepsia, the most common being acid-related dyspepsia which is associated with excess gastric acidity and may lead to peptic ulcers, gastroesophageal reflux disease (GERD), and other conditions characterized by excess gastric acidity. Functional dyspepsia (FD), is not associated with excess gastric acidity. Rather, the primary pathophysiological causative factor for FD is unclear.

FD is a visceral hypersensitivity state characterized by chronic or recurrent upper abdominal symptoms in the absence of any identifiable organic pathology, such as peptic ulceration, gastric cancer, chronic pancreatitis or GERD. The absence of identifiable organic pathology is established using conventional techniques including endoscopy, radiography, histology, and other techniques known to those skilled in the art.

The primary symptoms of FD include upper abdominal pain or discomfort (often aggravated by food or milk or occurring after meals), early satiety (which can lead to weight loss or anorexia), nausea and vomiting, bloating, belching, and post-prandial fullness.

FD has been divided into subtypes based upon the predominant symptom(s) observed in the patient. “Ulcer-like” FD is characterized primarily by pain. “Reflux-like” FD is primarily characterized by heartburn that is often alleviated by acid-suppressing agents. It is believed that most cases of reflux-like FD can actually be attributed to GERD, and is not actually FD because the condition can be associated with an organic pathology. “Dysmotility-like” FD is characterized primarily by discomfort, bloating, nausea, vomiting, early satiety, and post-prandial fullness. “Unspecified” FD refers to FD patients having symptoms that do not fit into the above categories. Typically FD patients exhibit symptoms across the various sub-types.

Several factors have been evaluated in an attempt to understand the pathophysiological mechanisms behind FD. Factors which have been investigated include gastrointestinal dysmotility, inflammation of the gastric or duodenal mucosa, eroded mucosal surfaces in the gastrointestinal tract, abnormal gastric emptying, and Helicobacter pylori infection. However, studies have revealed that no single factor or combination of factors is associated with all cases of FD.

The conventional treatment options for FD reflect the assumption that FD is attributable to the foregoing pathophysiological factors. The conventional treatment options for FD have proven to be of limited efficacy in many patients.

For example, only about 25-30 percent of FD patients present abnormal gastric emptying. Thus treatments designed to normalize gastric emptying have met with limited success in the treatment of FD. Prokinetic agents such as metoclopromide and cisapride have been tested for the ability to rectify dysmotility in FD patients. However only 25-30 percent of FD patients exhibit objective evidence of gastroparesis and only about 10 percent have small intestinal dysmotility; thus the correlation between dysmotility and FD is weak, as not all FD patients exhibit motility disturbances. In addition, both metoclopramide and cisapride have been associated with undesirable adverse effects which limit their application; cisapride having been recently limited in its use in the US.

Symptoms exhibited by many FD patients have traditionally been erroneously associated with acid-related dyspepsia (i.e., heartburn, reflux of acid, etc.) and as a result antisecretory agents have been evaluated for their applicability in the treatment of FD. Efficacy studies with histamine H-2 receptor antagonists have produced conflicting results; several studies having failed to demonstrate an effect different from placebo. Patients with ulcer-like FD or coexistent symptomatic GERD were the most likely to respond positively to treatment with an H-2 receptor antagonist.

It has been hypothesized that proton-pump inhibitors, which are commonly employed for acid-related dyspepsia, may benefit certain patients through the complete or near-complete inhibition of acid secretion, but no conclusive evidence supporting this hypothesis has been reported.

The efficacy of sucralfate in FD patients has been evaluated but again the results are inconclusive.

Finally, the eradication of H. pylori has been considered extensively with the general conclusion that H. pylori is not associated with FD and therefore its eradication is of limited value to FD patients.

Thus, unlike acid-related dyspepsia, current treatments for FD are limited and ineffective for the treatment of many FD patients.

The potential role of 5-HT3 receptor antagonists in FD has been considered with conflicting results. D. Maxton et al., Aliment Pharmacol. Ther. 10:595-599 (1996), concluded that a study with ondansetron justified evaluation of the therapeutic potential of selective 5-HT antagonists in both functional dyspepsia and irritable bowel syndrome. To the contrary, S. Klatt, et al., Digestion 60:147-152 (1999), concluded that another 5-HT antagonist, tropisetron did not influence gastric accommodation, reflex relaxation or gastric sensitivity in FD patients and healthy controls.

F. Zerbib, et al., Aliment Pharmacol. Ther. 8:403-407 (1994) reported that alosetron had an effect similar to placebo on the visceral perception scores in both isobarometric and isovolumetric distensions and concluded that 5-HT₃ receptors do not appear to be involved in the visceral perception of gastric distension in healthy subjects.

There remains a need for new methods for the treatment of FD.

### SUMMARY OF THE INVENTION

According to one aspect, the present invention provides a method for treatment of functional dyspepsia comprising administering an effective amount of alosetron or a pharmaceutically acceptable derivative thereof.

According to another aspect, the present invention provides a method of treatment for functional dyspepsia in females comprising administering an effective amount of alosetron or a pharmaceutically acceptable derivative thereof.

According to third aspect, the present invention provides a method of treatment for functional dyspepsia in subjects having normal gastric emptying comprising administering an effective amount of alosetron or a pharmaceutically acceptable derivative thereof.

According to a fourth aspect, the present invention provides the use of alosetron or a pharmaceutically acceptable derivative thereof in the manufacture of a medicament for the treatment of functional dyspepsia.

According to a fifth aspect, the present invention provides the use of alosetron or a pharmaceutically acceptable derivative thereof in the manufacture of a medicament for the treatment of functional dyspepsia in females.

According to a sixth aspect, the present invention provides the use of alosetron or a pharmaceutically acceptable derivative thereof in the manufacture of a medicament for the treatment of functional dyspepsia in subjects having normal gastric emptying.

### DESCRIPTION OF THE PREFERRED EMBODIMENTS

By “pharmaceutically acceptable derivative” is meant any pharmaceutically acceptable salt or solvate of alosetron, which upon administration to the recipient is capable of providing (directly or indirectly) alosetron or an active metabolite or residue thereof. Suitable pharmaceutically acceptable salts of alosetron include acid addition salts formed with inorganic or organic acids (for example hydrochlorides, hydrobromides, sulphates, phosphates, benzoates, naphthoates, hydroxynaphthoates, p-toluenesulphonates, methanesulphonates, sulphamates, ascorbates, tartrates, salicylates, succinates, lactates, glutarates, glutaconates, acetates, tricarballylates, citrates, fumarates and maleates), and solvates (for example hydrates) thereof.

In a preferred embodiment of the present invention alosetron is employed in the form of its hydrochloride salt.

It is to be understood that reference to treatment includes both treatment of established symptoms and prophylactic treatment, unless explicitly stated otherwise.

Conveniently, alosetron or a pharmaceutically acceptable derivative thereof may be formulated in conventional manner using one or more pharmaceutically acceptable carriers or excipients. Thus alosetron or a pharmaceutically acceptable derivative thereof may, for example, be formulated for oral, sub-lingual, buccal, parenteral, rectal or intranasal administration, or in a form suitable for administration by inhalation or insufflation (either through the mouth or nose), or in a form suitable for topical administration,

For oral administration the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrates (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl-p-hydroxybenzoates or sorbic acid).

For buccal administration the compositions may take the form of tablets or lozenges formulated in conventional manner.

For parenteral administration the compositions may take the form of injections, conveniently intravenous, intramuscular or subcutaneous injections, for example bolus injections or continuous intravenous infusions. Formulations for injection may be presented in unit dosage form e.g. in ampoules or in multi-dose containers, optionally with an added preservative.

The compositions for parenteral administration may take such forms as suspensions, solutions or emulsions in oily or aqueous vehicles, and may contain formulatory agents such as suspending, stabilising and/or dispersing agents. Alternatively, the compositions may be in dry form such as a powder, crystalline or freeze-dried solid for constitution with a suitable vehicle, e.g. sterile pyrogen-free water or isotonic saline before use. They may be presented, for example, in sterile ampoules or vials.

For rectal administration the compositions may take the form of suppositories or retention enemas.

Tablets for sub-lingual administration may be formulated in a conventional manner. For intranasal administration, or administration by inhalation or insufflation, conventional formulations may be employed.

For topical administration the pharmaceutical compositions may be liquids, for example solutions, suspensions or emulsions presented in the form of creams or gels. In addition to the formulations described previously, the compositions may also be formulated as a depot preparations. Such long acting formulations may be administered by implantation (for example subcutaneously, transcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

It will be appreciated that the precise therapeutic dose of alosetron, expressed in the form of its free base, will depend on the age and condition of the patient and the nature of the FD to be treated, and will be at the ultimate discretion of the attendant physician.

However, in general, effective doses for the treatment of FD, the treatment of FD in females, and the treatment of FD in subjects with normal gastric emptying will lie in the range of 0.001 to 500 mg, such as 0.01 to 100 mg, preferably 0.05 to 50 mg, for example 0.5 to 25 mg per unit dose, which could be administered in single or divided doses, for example, 1 to 4 times per day.

In a preferred embodiment, effective doses of alosetron for the treatment of FD, the treatment of FD in females, and the treatment of FD in subjects with normal gastric emptying will lie in the range of 0.01 to 100 mg, such as 0.05 to 50mg, preferably 0.1 to 25 mg, for example 0.5, 1, 2 or 4 mg of alosetron per unit dose, which could be administered in single or divided doses, for example, 1 to 4 times per day. In a preferred embodiment, 0.5 mg of alosetron is administered twice per day. In another preferred embodiment, 1 mg of alosetron is administered twice per day. In another preferred embodiment, 2 mg of alosetron is adminstered once per day.

The use of alosetron in the treatment of FD is supported by the following clinical study.

Patients

Three hundred and twenty (320) ambulatory outpatient male and female FD patients at least 18 years of age who have chronic or recurrent abdominal pain or discomfort centered in the upper abdomen were randomised for study comprising twelve (12) weeks of treatment: eighty one (81) were randomised to treatment with placebo BID, seventy seven (77) to 0.5 mg BID alosetron, seventy nine (79) to 1 mg BID alosetron, and eighty three (83) to 2 mg BID alosetron.

Study participants included patients who have been diagnosed with FD and patients with symptoms that fulfilled the modified Rome criteria for FD for at least 6 months. Study participants were screened for two weeks prior to the testing phase, to confirm symptoms of sufficient severity for participation in the study. During the two-week screening period, those patients enrolled from the U.S. underwent a 90-minute gastric emptying test to characterize their gastric motility. At the end of the two-week period, all study participants underwent an esophagogastroduodenoseope (EGD) to rule out organic disease and obtain a biopsy for H. pylori within one month of screening.

Study Design

The study was double-blinded and placebo-matched. Eligible subjects were randomized (1:1:1:1 ratio) to either alosetron 0.5 mg, 1 mg, 2 mg, or matched placebo twice daily for a 12-week treatment phase followed by a one-week follow-up period. Laboratory evaluations and adverse events were recorded at the 4, 8 and 12-week (or final) treatment visits.

The study was designed to elicit measurements relating to efficacy and safety. The primary endpoint was adequate relief of FD (upper abdominal) pain or discomfort. Secondary endpoints included 1) changes in daily upper abdominal pain severity ratings, 2) proportion of pain free days, 3) changes in self-ratings of nausea, early satiety, bloating or distension, post-prandial fullness and burping or belching, 4) changes in quality of life (QOL) SF-36 scores, 5) changes in SCL-90R® scores, and 6) safety and tolerability with respect to the incidence of adverse events and changes in laboratory values.

Daily and weekly symptom data were collected using an electronic touch-tone telephone based system described in J. Harding, et al., Gastroenterology 112:A745 (1997); and J. Harding, et al., Aliment Pharm. Ther. 11:1073-761997.

Severity of abdominal pain, nausea and bloating were assessed on a daily basis using a five point scale (0=none; 1=very mild; 2=mild; 3=moderate; 4=severe). During screening, the pain severity score was required to average ≧1.5 for entry into the study. During the treatment and follow-up phases, subjects were asked weekly if they had obtained adequate relief of their upper abdominal pain or discomfort during the previous 7 days. Subjects also responded daily to yes/no questions assessing whether early satiety, post-prandial fullness and burping or belching were present.

Statistics

For this study, a “completed subject” is one who completed the screening phase, treatment phase and one-week follow-up phase of the study. Subjects who did not complete the three phases of the study were considered prematurely discontinued and were not replaced.

The sample size was chosen with 90% power at the α=0.05 level of significance. The number of subjects per treatment group needed to detect a 20% difference in proportion of patients with adequate relief between alosetron and any dose of alosetron was 64 per group, assuming the proportion with adequate relief in the placebo group was 35%. A target sample size of 70 patients per treatment group was chosen to allow for an approximate 10% dropout rate.

All statistical analyses were performed using two-sided significance tests at the 0.05 level. The experiment wise type I error rate was controlled at the 0.05 significance level using a procedure based on sequential significance testing and the principle of closed tests. Treatment-by-center interactions were assessed by pooling centers a priori into geographical clusters, and using the appropriate statistical test stratified by these clusters.

Analyses were by Intent-to-Treat (ITT) using the last observation carried forward (LOCF) method for handling missing data. The ITT population included all subjects randomized to study treatment. The primary efficacy endpoint was the adequate relief of upper abdominal pain or discomfort. The 12-week average rate of adequate relief was calculated as the sum of the number of responses of “yes” over the 12 weeks of the treatment phase divided by 12 and multiplied by 100 using the LOCF approach. The proportion of patients with weekly adequate relief of upper abdominal pain or discomfort was compared between treatment groups using the Mantel-Haenszel test stratified by geographical clusters of treatment centers.

Secondary efficacy measures included subject self-rating of pain severity, frequency and the FD symptoms of nausea, bloating/distension, burping/belching, early satiety and post-prandial fullness. The percentage of pain-free days, days with nausea, bloating/distension, early satiety, post-prandial fullness and burping/belching were calculated at baseline (for the 2 week screening period), and for each week of the treatment and follow-up phases. In addition, average severity of pain, nausea and bloating/distension were calculated at baseline and for the same weekly intervals. For each parameter, changes from baseline in treatment groups were compared using a Wilcoxon rank sum test stratified by geographical cluster.

Primary and secondary efficacy measures was summarized by subgroups consisting of gender, age race, hormone use, H. pylori status, FD subtype, and gastric emptying status.

To validate adequate relief of upper abdominal pain or discomfort and describe its relationship to other efficacy parameters, the correlation between adequate relief of upper abdominal pain or discomfort and proportion of pain-free days, pain severity, nausea, early satiety, bloating/distension, burping/belching and post-prandial fullness, were assessed weekly using Spearman's Correlation Coefficient irrespective of treatment. The adequate relief response groups (yes/no) were compared with respect to other efficacy parameters at each week using a Wilcoxon rank-sum test.

The proportion of subjects reporting adequate upper abdominal pain or discomfort relief were compared between treatment groups at each week during treatment and follow-up, LOCF and observed, using a Mantel-Haenszel test stratified by cluster.

Average Weekly Adequate Relief of Pain and Discomfort by Gender

| | |
|---|---|
| Adequate Relief | Alosetron (mg) BID |

| Rates | 0 (Placebo) | 0.5 | 1 | 2 |
|---|---|---|---|---|
| Females (n = 50-60) | 40 | 49 | 54 | 43 |
| Males (n = 20-33) | 39 | 39 | 43 | 40 |

Examination of each dose of alosetron showed a greater adequate relief rate as compared with placebo.

Average Weekly Adequate Relief by Gastric Emptying

| | |
|---|---|
| % with Adequate | Alosetron (mg) BID |

| Relief | 0 (Placebo) | 0.5 | 1 | 2 |
|---|---|---|---|---|
| Normal (n = 32-34) | 38 | 60 | 56 | 43 |
| Abnormal (n = 14-16) | 66 | 51 | 59 | 45 |

Improvement was observed for all doses in both subjects with normal gastric emptying and subjects with abnormal gastric emptying.

To validate adequate relief of upper abdominal pain or discomfort and describe its relationship to the other efficacy parameters, the correlation between adequate relief of upper abdominal pain or discomfort and the other efficacy parameters (i.e., proportion of pain-free days, pain severity, nausea, early satiety, bloating/distension, burping/belching and post-prandial fullness) were assessed using Spearman's Correlation Coefficient. The correlation was irrespective of treatment, and was performed each week using the weekly assessments. In addition, the adequate relief response groups were compared with respect to the other efficacy parameters at each week using a Wilcoxon rank-sum test.

Adequate Relief Correlations

| | Responders* | Nonresponders | P |
|---|---|---|---|
| Pain Severity | −1.7 | −0.6 | <0.001 |
| Early Satiety | −42.1% | −16.3% | =0.001 |
| Bloating/Distension | −34.1% | −6.1% | <0.001 |

*Correlation at week 12.

The effects of gender, age, race, hormone use, H. pylori, subtype of FD and gastric emptying status on adequate relief and pain or discomfort-free days was assessed as secondary analyses.

Demographic and Baseline Characteristics of the
Patients in All Four Treatment Groups

| | Alosetron (mg) BID | | | |
|---|---|---|---|---|
| | 0 | | | |
| | (Placebo) | 0.5 | 1 | 2 |
| Age | 44.2 | 45.4 | 46.1 | 47.0 |
| Race-white | 83% | 91% | 81% | 87% |
| Gender-Female | 74% | 66% | 75% | 60% |
| Gender-Male | 26% | 34% | 25% | 40% |
| Time Since FD Symptoms (yrs) | 6.60 | 7.99 | 6.70 | 6.97 |
| Gastric Emptying-Normal | 68% | 70% | 69% | 71% |
| Gastric Emptying-Abnormal | 32% | 30% | 31% | 29% |

Adverse Events

Adverse events were coded and group by body system. Adverse events occurring after randomization were summarized separately from adverse events occurring during the screening phase. For each body system and over all body systems, the frequency of subjects reporting at least one adverse event was calculated by treatment group. In addition, the number of subjects reporting at least one drug-related adverse event and the number of subjects reporting serious adverse events were summarized by treatment group. The four treatment groups were compared using a correlation chi-square test; pairwise comparisons were made with Fisher's exact test.

Adverse Events

Alosetron (mg) BID

| | 0 | 0.5 | 1 | 2 |
|---|---|---|---|---|
| Any event | 51% | 68% | 68% | 75% |
| GI event | 24% | 58% | 52% | 63% |
| −constipation | 13% | 45% | 34% | 46% |
| Neurology | 14% | 9% | 4% | 16% |

### SUMMARY

The results demonstrate that alosetron substantially improved abdominal pain and discomfort in FD patients, particularly female FD patients. Alosetron also substantially improved nausea, early satiety and post-prandial fullness in female patients and patients with normal gastric emptying.

Based upon the results of the present study, alosetron is an effective and well-tolerated therapy for the treatment of FD patients. In particular, the use of alosetron for the treatment of functional dyspepsia shows unexpected benefits in female patients.

1. A method for treatment of functional dyspepsia comprising administering an effective amount of alosetron or a pharmaceutically acceptable derivative thereof.

2. The method of treatment according to claim 1 wherein alosetron is in the form of its hydrochloride salt.

3. A method of treatment for functional dyspepsia in females comprising administering an effective amount of alosetron or a pharmaceutically acceptable derivative thereof.

4. The method of treatment according to claim 3, wherein alosetron is in the form of its hydrochloride salt.

5. A method of treatment for functional dyspepsia in subjects having normal gastric emptying comprising administering an effective amount of alosetron or a pharmaceutically acceptable derivative thereof.

6. The method of treatment according to claim 5, wherein alosetron is in the form of its hydrochloride salt.

7. Use of alosetron or a pharmaceutically acceptable derivative thereof in the manufacture of a medicament for the treatment of functional dyspepsia.

8. Use of alosetron or a pharmaceutically acceptable derivative thereof in the manufacture of a medicament for the treatment of functional dyspepsia in females.

9. Use of alosetron or a pharmaceutically acceptable derivative thereof in the manufacture of a medicament for the treatment of functional dyspepsia in subjects having normal gastric emptying.

10. Use according to any of claims 7, 8 or 9 wherein alosetron is in the form of its hydrochloride salt.
